(19) United States
(12) Patent Application Publication
(10) Pub. No.: US 2012/0283282 A1
(43) Pub. Date: Nov. 8, 2012

(54) Photoproducts of Tryptophan, Their Synthesis and Uses Thereof

(76) Inventors: Arleen Rifkind, New York, NY (US); Silvia Diani-Moore, Roosevelt Island, NY (US); J. David Warren, New York, NY (US); Erin Hall, Fort Mitchell, KY (US); Yuliang Ma, New York, NY (US)

(21) Appl. No.: 13/379,327

(22) PCT Filed: Jun. 18, 2010
(86) PCT No.: PCT/US10/39248

§ 371 Date: Feb. 29, 2012

Related U.S. Application Data
(60) Provisional application No. 61/218,338, filed on Jun. 18, 2009.

(51) Int. Cl. A61K 31/437; C12N 5/071; A61P 3/00; A61K 31/4745; C07D 471/04; A61P 35/00

(52) U.S. Cl. 514/280; 546/87; 514/292; 435/375

(57) ABSTRACT

We have found that exposure of an aqueous tryptophan solution to window sunlight results in the production of multiple tryptophan photoproducts that have the capability of activating the aryl hydrocarbon receptor and increasing the production of AhR target genes and proteins in hepatocytes. We have isolated three of those photoproducts not previously identified as AhR activators, their chemical identification and synthesis and the demonstration that all three have biologic activities as novel AhR activators. Further, one of the three is a completely novel, not previously described, chemical compound.

This invention was made with Government support under Grant Number ES03606 awarded by the National Institutes of Health. The United States Government has certain rights in the invention.

### BACKGROUND

Thus far the major activators of the AhR have been recognized to be toxic chemicals of which TCDD (Dioxin, 2,3,7,8-tetrachlorodibenzo-p-dioxin) and planar polychlorinated biphenyls (PCBs), benzo(a)pyrene are prototypes. Activation of the AhR by these agents results in major toxic effects: a wasting syndrome, immune system toxicity, cancer, death. There is increasing evidence that there are other more naturally occurring AhR activators to which people can be commonly exposed in the environment. A major group includes indole derivatives in food products (probably tryptophan derivatives) and tryptophan photoproducts generated by UV light. It is a significant biologic and health related question whether these other AhR ligands cause toxicities or not. A group from Sweden, headed by Agneta and Rannug, have described several photoproducts of tryptophan that activate the AhR, different from ours. They have highlighted the significance of one product, FICZ.

Our data show (a) that tryptophan is made into multiple photoproducts that can activate the AhR (b) that FICZ is not the only one nor the major constituent of the mixture of tryptophan photoproducts that activates the AhR, and (c) AhR activated tryptophan products can be generated by ordinary exposures to light in the work and home environment (i.e. sunlight through window glass and fluorescent bulbs).

### SUMMARY

Novel compositions are provided. Methods to use novel compounds as well as known compounds to modulate the AhR are provided. Methods to treat diseases related to cell proliferation and metabolic conditions are provided.

### BRIEF DESCRIPTION OF THE DRAWINGS

FIG. 1. Structure of Compound F4

FIG. 1. Structure of Compound F5

FIG. 1. Structure of Compound F7

FIG. 4. Potency and Efficacy of F7: comparison with TCDD and FICZ

FIG. 5. F7 decreases hepatic glucose output

FIG. 6. Exposure of aqueous tryptophan solution to window sunlight

FIG. 7. Activation of Tryptophan by different light sources

FIG. 8. Sustained CYP1A induction by sunlight-activated tryptophan (aTRP)

FIG. 9. Identification of TRP photoproducts: Separation by RP-HPLC

FIG. 10. Identification of TRP photoproducts: Separation by RP-HPLC

FIG. 11. Identification of a main CYP1A inducing peak in Fraction 7

FIG. 12. LC coupled with TOF mass spectrometer: Extracted ion chromatogram for Fraction7

FIG. 13. Molecular formula generation—F7

FIG. 14. Identity of Compound F7, an Major Component in Fraction7=1-(1H-indol-3-yl)-9H-pyrido[3,4-b]indole

FIG. 15 MS spectrum F7

FIG. 16 Q-TOF MS/MS Spectra of Protonated Compound F7 (synthetic std) at Different Collision Energy (15-40 eV)

FIG. 17 Comparison of MS/MS Spectra of the [M+H]+Ions (m/z 284.12) for Both Synthetic and Purified Compound F7

FIG. 18 MS/MS Spectrum of the m/z 140.05 ion

FIG. 19 Scheme 1. Formation of the major fragment ions m/z 115, 167 and 140 and their further fragmentation ions from compound F7

FIG. 20. Identity of a New Compound (F4) from Fraction 4,3-((9H-pyrido[3,4-b]indol-1-yl)methyl)indolin-2-one

FIG. 21 MS/MS Spectrum of Protonated Compound F4

FIG. 22. Scheme 2A. Structure Elucidation of Compound F4, a new tryptophan-related photoproduct

FIG. 23 Hypothetical pathways from tryptophan to F4 and F7 after exposure to sunlight

FIG. 24 Formation of F7: Hypothetical pathway from tryptophan after exposure to sunlight

FIG. 25 LC coupled with TOF mass spectrometer: Extracted ion chromatograms for Fractions 4 and 5

FIG. 26 Molecular formula generation—F4 and F5

FIG. 27 Structures for F4 and F5

FIG. 28 Synthetic route to F4 and analogs

FIG. 29 3D Structures of the three compounds (Chemdraw)

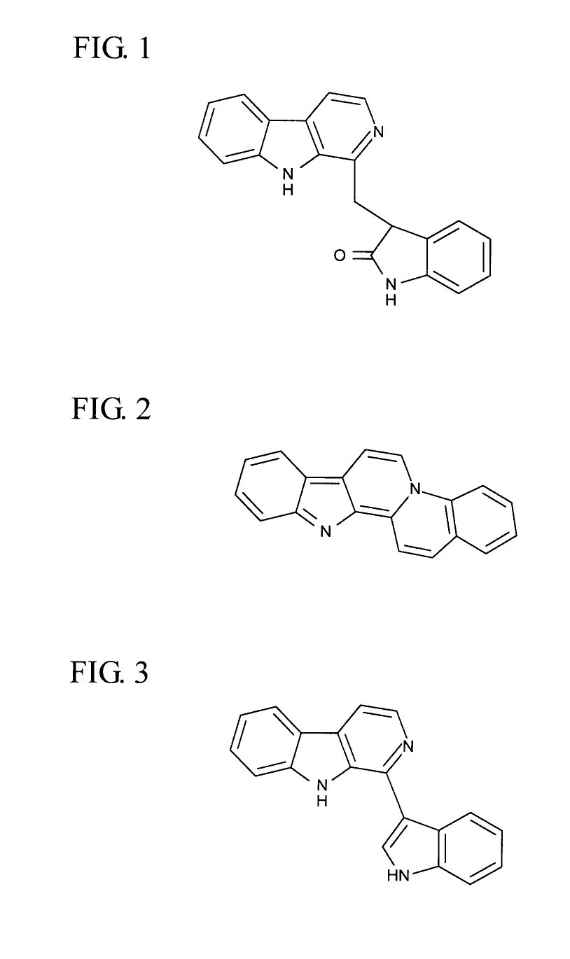

### DETAILED DESCRIPTION

In the following description, reference is made to the accompanying drawings that form a part hereof, and in which is shown by way of illustration specific embodiments which may be practiced. These embodiments are described in detail to enable those skilled in the art to practice the invention, and it is to be understood that other embodiments may be utilized and that structural, logical and electrical changes may be made without departing from the scope of the present invention. The following description of example embodiments is, therefore, not to be taken in a limited sense, and the scope of the present invention is defined by the appended claims.

The Abstract is provided to comply with 37 C.F.R. §1.72(b) to allow the reader to quickly ascertain the nature and gist of the technical disclosure. The Abstract is submitted with the understanding that it will not be used to interpret or limit the scope or meaning of the claims.

Photooxidized tryptophan (TRP) in tissue culture medium elicits a transient cytochrome P450 (CYP1) induction response in cultured cells. We have shown that exposure of TRP to window sunlight (aTRP) greatly increased the potency, efficacy, and duration of CYP1A induction by TRP in primary chick embryo hepatocytes and in vivo. Aqueous TRP exposed to sunlight for 7 days exhibited a 100-fold or greater increase in potency over TRP in medium. The induction response was sustained for at least 48 h and was comparable in efficacy to 2,3,7,8-tetrachlorodibenzo-p-dioxin. In hepatocytes, increases in mRNAs for CYP1A4 and CYP1A5, chick orthologs of mammalian CYP1A1 and 1A2, preceded increases in CYP1A proteins and enzyme activities, 7-ethoxyresorufin deethylase (EROD) for CYP1A4 and arachidonic acid epoxygenation for CYP1A5, consistent with a transcriptional mechanism. Aryl hydrocarbon receptor (AhR) dependence was evidenced by aTRP induction of EROD in wild-type Hepa1c1c7 cells but not in AhR-defective (c35) mutants. Preparations of aTRP were stable for many months at 4° C. and were relatively resistant to metabolism by hepatocytes or liver microsomes. Fractionation of aTRP by HPLC analysis coupled with EROD assays showed that aTRP contained multiple photoproducts and CYP1A inducing components, which varied in sensitivity to metabolism by hepatocytes. The previously identified TRP photoproduct, 6-formylindolo[3,2-b]carbazole (FICZ), was one component, but FICZ was not required for CYP1A induction by the aTRP mixture. These findings identify the indoor environment, and window sunlight in particular, as a new source of CYP1A inducers. Further, the evidence that biologically active metabolites of an endogenous substrate, arachidonic acid, are formed by aTRP-induced CYP1A provides a pathway by which TRP photoproducts, like toxic xenobiotics, could have significant physiologic effects.

Fourteen fractions obtained by separation of sunlight-activated tryptophan products by reverse phase (RP)-HPLC all exhibited CYP1A inducing capacity, showing that sunlight-activated tryptophan contained multiple AhR-activating compounds. Further, each fraction contained multiple UV absorbing peaks. We have now identified the chemical composition of the main inducing components in three of the fractions with high CYP1A inducing capacity. Each of the fractions was subjected to serial RP-HPLC separations with different gradients and flow rates to segregate the peaks. A single peak responsible for the majority of the CYP1A inducing activity in each fraction was isolated. An accurate mass for each of those tryptophan photoproducts was determined using an Agilent 6220 accurate-mass time-of-flight (TOF) liquid chromatography/mass spectrometry (LC/MS) system equipped with a dual electrospray source. The structures were elucidated by a detailed interpretation of the collision-induced dissociation (CID) product ion spectra obtained using an Agilent 6520 accurate-mass quadrupole time-of-flight (Q-TOF) tandem mass spectrometer. The routes by which the predicted structures might be derived from tryptophan were traced. The proposed products were chemically synthesized and tested for CYP1A induction capacity. Their potencies and efficacies were compared to those of TCDD and the known tryptophan photoproduct and AhR ligand, 6-formylindolo[3,2-b]carbazole (FICZ). The identification of the inducing compounds and their relative activities are presented.

These compounds modulate the AhR receptor. AhR is a conserved basic helix-loop-helix ligand activated transcription factor. It is a cytosolic transcription factor that is normally inactive, bound to several co-chaperones. Upon ligand binding, the chaperones dissociate resulting in AhR translocating into the nucleus and dimerizing with ARNT (AhR nuclear translocator), leading to changes in gene transcription.

AhR is activated by a variety of ligands, synthetic or naturally occurring. Naturally occurring compounds that have been identified as ligands of AHR include derivatives of tryptophan such as indigo and indirubin, tetrapyroles such as bilirubin, lipoxin A4, prostaglandin G, modified low-density lipoprotein and several dietary carotinoids. Synthetic compounds include members of the halogenated aromatic hydrocarbons and polycyclic aromatic hydrocarbons.

AhR is known to be involved in regulating the cell cycle. Therefore compounds that modulate AhR have utility for diseases and conditions involving cell profileration and hyperplasia, including but not limited to cancers, psoriasis, warts, and other conditions.

Further, AHR is known to suppress gluconeogenesis via PGC-1. (Rifkind et al, in press). As per Yoon (Yoon J C, et al. Nature 413: 131-8, 2001) the suppression of gluconeogenesis and hepatic glucose output remains a very attractive therapeutic target in diabetes. The anti-diabetic drug metformin is thought to work through the suppression of hepatic glucose output32, but very little is known about its mechanism. Suppressing PGC-1 function in the liver without compromising its effects in other non-gluconeogenic tissues such as brown fat and muscle could yield medically significant anti-diabetic effects. Compositions that prevent suppression of gluconeogenesis may be useful to treat wasting diseases, such as AIDs-related wasting, cachexia accompanying cancer or chemotherapy, and dioxin poisoning.

### DEFINITIONS

Hetero- denotes a compound or substituent or group containing a heteroatom. A heteroatom is any atom that is not carbon or hydrogen; it typically, but not exclusively, denotes nitrogen, oxygen, sulfur, phosphorus, boron, chlorine, bromine, flourine, or

An alkyl group is branched or unbranched and contains 1 to 7 carbon atoms, preferably 1-4 carbon atoms. Lower alkyl represents; for example, methyl, ethyl, propyl, butyl, isopropyl, isobutyl, or tertiary butyl.

An alkene, alkenyl or alkenyloxy group is branched or unbranched and contains 2 to 7 carbon atoms, preferably 1-4 carbon atoms and contains at least one carbon-carbon double bond. Lower alkene lower alkenyl or lower alkenyloxy represents for example vinyl, prop-1-enyl, allyl, butenyl, isopropenyl or isobutenyl and the oxy equivalents thereof.

An alkyne, alkynyl or alkynyloxy group is branched or unbranched and contains 2 to 7 carbon atoms, preferably 1-4 carbon atoms and contains at least one carbon-carbon triple bond. Lower alkyne or alkynyl represents for example ethynyl, prop-1-ynyl (propargyl), butynyl, isopropynyl or isobutynyl and the oxy equivalents thereof.

Aryl represents carbocyclic or heterocyclic aryl.

Carbocyclic aryl represents monocyclic, bicyclic or tricyclic aryl, for example phenyl or phenyl mono-, di- or tri-substituted by one, two or three radicals selected from lower alkyl, lower alkoxy, aryl, hydroxy, halogen, cyano, trifluoromethyl, lower alkylenedioxy and oxy-C.sub.2-C.sub.3-alkylene; or 1- or 2-naphthyl; or 1- or 2-phenanthrenyl. Lower alkylenedioxy is a divalent substituent attached to two adjacent carbon atoms of phenyl, e.g. methylenedioxy or ethylenedioxy. Oxy-C.sub.2-C.sub.3-alkylene is also a divalent substituent attached to two adjacent carbon atoms of phenyl, e.g. oxyethylene or oxypropylene. An example for oxy-C.sub.2-C.sub.3-alkylene-phenyl is 2,3-dihydrobenzofuran-5-yl.

Heterocyclic aryl represents monocyclic or bicyclic heteroaryl, for example pyridyl, indolyl, quinoxalinyl, quinolinyl, isoquinolinyl, benzothienyl, benzofuranyl, benzopyranyl, benzothiophenyl, benzothiopyranyl, furanyl, pyrrolyl, thiazolyl, oxazolyl, isoxazolyl, triazolyl, tetrazolyl, pyrazolyl, imidazolyl, thienyl, or any said radical substituted, especially mono or di-substituted as defined above. Preferably, heterocyclic aryl is thiophenyl, tetrahydrothiophenyl, thienopyridinyl (e.g. thieno[3,2-c]pyridinyl), benzothiophenyl (e.g. benzo)[b]thiophenyl), pyrrolyl, pyridyl, indolyl, quinolinyl, imidazolyl, or any said radical substituted, especially mono- or di- or trisubstituted as defined below.

Cycloalkyl represents a saturated cyclic hydrocarbon optionally substituted by lower alkyl which contains 3 to 10 ring carbons and is advantageously cyclopentyl, cyclohexyl, cycloheptyl or cyclooctyl optionally substituted by lower alkyl.

Heterocycloalkyl represents a mono-, di- or tricyclic moiety comprising from 3 to 18 ring atoms, at least one of which (e.g. from 1 to 3 ring atoms) is a hetero atom selected from O, S or N, and the remaining ring atoms are carbon atoms, which are saturated or comprise one or more unsaturated alkenyl or alkynyl bonds. Preferred heterocycloalkyl moieties are N-heterocycloalkyl moieties containing from 5 to 7 ring atoms and optionally containing a further hetero atom, selected from O, S or N. Heterocycloalkyl may be substituted, for instance, as hereinafter defined and including .dbd.O substitution on the heterocyclic ring e.g. as pyrrolidinone. Examples of preferred heterocycloalkyl moieties are pyrrolidine, tetrahydrothiophene, tetrahydrofuran, piperidine, pyran, dioxane, morpholino, or piperazine, especially piperidine, morpholino or piperazine.

Pharmaceutical Preparations

The present invention also provides a method for the prevention or treatment of a disease characterized by need for AhR modulation in a subject, by administering to the subject a composition comprising a therapeutically effective amount of an modulator of AhR and a pharmaceutically acceptable excipient.

In another aspect, the present invention provides pharmaceutically acceptable compositions which comprise a therapeutically-effective amount of one or more of the modulators of AhR, as described above, formulated together with one or more pharmaceutically acceptable excipients. In another aspect, the present invention provides pharmaceutically acceptable compositions which comprise a therapeutically-effective amount of one or more of the modulators of AhR, as described above, formulated together with one or more pharmaceutically acceptable excipients and other therapeutically effective medications known in the art allowing for but not limited to combination therapies to improve overall efficacy of each individual therapeutic or to limit the concentration of either therapeutic to avoid side effects and maintain efficacy. The active ingredient and excipient(s) may be formulated into compositions and dosage forms according to methods known in the art. As described in detail below, the pharmaceutical compositions of the present invention may be specially formulated for administration in solid or liquid form, including those adapted for the following: (1) oral administration, for example, tablets, capsules, powders, granules, pastes for application to the tongue, aqueous or non-aqueous solutions or suspensions, drenches, or syrups; (2) parenteral administration, for example, by subcutaneous, intramuscular or intravenous injection as, for example, a sterile solution or suspension; (3) topical application, for example, as a cream, ointment or spray applied to the skin, lungs, or mucous membranes; or (4) intravaginally or intrarectally, for example, as a pessary, cream or foam; (5) sublingually or buccally; (6) ocularly; (7) transdermally; or (8) nasally.

The phrase “pharmaceutically acceptable” is employed herein to refer to those compounds, materials, compositions, and/or dosage forms which are, within the scope of sound medical judgment, suitable for use in contact with the tissues of the subject with toxicity, irritation, allergic response, or other problems or complications, commensurate with a reasonable benefit/risk ratio.

The phrase “pharmaceutically-acceptable excipient” as used herein refers to a pharmaceutically-acceptable material, composition or vehicle, such as a liquid or solid filler, diluent, carrier, manufacturing aid (e.g., lubricant, talc magnesium, calcium or zinc stearate, or steric acid), solvent or encapsulating material, involved in carrying or transporting the therapeutic compound for administration to the subject. Each excipient should be “acceptable” in the sense of being compatible with the other ingredients of the formulation and not injurious to the subject. Some examples of materials which can serve as pharmaceutically-acceptable excipients include: sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations. If desired, certain sweetening and/or flavoring and/or coloring agents may be added. Other suitable excipients can be found in standard pharmaceutical texts, e.g. in “Remington's Pharmaceutical Sciences”, The Science and Practice of Pharmacy, 19th Ed. Mack Publishing Company, Easton, Pa., (1995).

Excipients are added to the composition for a variety of purposes. Diluents increase the bulk of a solid pharmaceutical composition, and may make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the subjects's stomach may be increased by the addition of a disintegrant to the composition. Disintegrants include alginic acid, carboxymethylcellulose calcium, carboxymethylcellulose sodium (e.g. Ac Di Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

In liquid pharmaceutical compositions of the present invention, the modulator of AhR and any other solid excipients are dissolved or suspended in a liquid carrier such as water, water-for-injection, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Flavoring agents and flavor enhancers may make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol and tartaric acid.

Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the present invention, a liquid composition may also contain a buffer such as guconic acid, lactic acid, citric acid or acetic acid, sodium guconate, sodium lactate, sodium citrate or sodium acetate. Selection of excipients and the amounts used may be readily determined by the formulation scientist based upon experience and consideration of standard procedures and reference works in the field.

Solid and liquid compositions may also be dyed using any pharmaceutically acceptable colorant to improve their appearance and/or facilitate patient identification of the product and unit dosage level.

The dosage form of the present invention may be a capsule containing the composition, for example, a powdered or granulated solid composition of the invention, within either a hard or soft shell. The shell may be made from gelatin and optionally contain a plasticizer such as glycerin and sorbitol, and an opacifying agent or colorant.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling may include any of the aforementioned blends and granulates that were described with reference to tableting, however, they are not subjected to a final tableting step.

### EXAMPLES

Tissue Culture Media Used for Primary Hepatocytes.

(1) Std. Ham's-9.18 g of Basal Medium Eagle (BME) (Cellgro, Mediatech Herndon, Va.) and 2.2 g of NaHCO3 were dissolved in 900 ml of distilled water. Additions were: 20 ml of 50× MEM essential amino acid solution containing 36 mM L-arginine, 10 mM L-cystine, 13.5 mM L-histidine, 20 mM L-isoleucine, 20 mM L-leucine, 25 mM L-lysine, 5 mM L-methionine, 10 mM L-phenylalanine, 20 mM L-threonine, 10 mM L-tyrosine, and 20 mM L-valine, 2.5 mM L-tryptophan, 200,000 U/penicillin-streptomycin, 20 ml of 100× MEM vitamin solution, 5 ml of 100× MEM nonessential amino acids, and 0.1 ml (10 mg/ml) of d-Biotin. pH was adjusted to 7.3-7.5, FBS added to a final concentration of 2%, and the total volume brought to 1 l with distilled water. (2) TRP-free medium—TRP-free Basal Medium Eagle (BME) was custom-prepared by Specialty Media (Phillipsburg, N.J.). Additions were the same as for Std. Ham's, except that TRP was excluded from the MEM essential amino acid solution.

Treatment of Hepatocytes.

β-Naphthoflavone (β-NF) in dimethylsulfoxide (DMSO) (10 mM) was diluted in TRP-free medium to 1 or 10 μM, the concentrations used in culture. TCDD (1.5 mM) in dioxane (J. T. Baker, Phillipsburg, N.J.) was diluted in dioxane to 1.5 μM and further diluted in TRP-free medium to 1 nM, a maximal CYP1A-inducing concentration, for addition to hepatocytes. Equivalent amounts of DMSO or dioxane were used as controls (final concentrations <0.1%). For treatment of hepatocytes, compounds were diluted and added to cells in TRP-free medium. Triplicate wells were used for each treatment.

EROD.

(1) In cultured hepatocytes—24-well plates—Medium was removed, cells washed with phosphate buffered saline (PBS; Cellgro by Mediatech), and 0.5 ml of the EROD reaction mixture added to each well (4 μM 7-ethoxyresorufin (7-ER) and 10 μM dicumarol in Std. Ham's). After 30 min at 37° C., two 0.2-ml aliquots were removed per well, 0.25 ml of cold acetone was added to each followed by centrifugation at 1400×g for 15 min. Resorufin fluorescence was measured in a Perkin Elmer MPF 3 spectrophotofluorimeter (Excitation (Ex) and Emission (Em)λ, 558 nm and 590 nm, respectively), using a quinine sulfate standard previously calibrated against resorufin. Emission spectra were obtained for selected samples to confirm the presence of a resorufin peak at 590 nm. Results for 24-well plates are given as pmol resorufin/well. 96-well plates—Medium was removed, cells washed with PBS, and 0.08 ml of the EROD reaction mixture described above added to each well. After 30 min at 37° C., resorufin fluorescence was read on a SpectraFluor fluorescence plate reader (Tecan, Durham, N.C.) at Ex and Em λ, 535 nm and 595 nm, respectively. A standard curve for resorufin (4.8 nM to 4.8 μM) added to wells of nontreated cultured hepatocytes immediately before reading was included in each experiment. Results for 96-well plates are given as pmol resorufin/ml. (2) In liver microsomes and hepatocyte homogenates—Reaction mixtures (0.24 ml) contained 15 to 30 μg chick embryo liver microsomal protein or 200 μg of hepatocyte homogenate protein in 0.039 M Tris-phosphate, pH 8.3, with 1.25 mM EDTA and 1 mg/ml BSA, 7-ER (4 μM unless otherwise indicated), and for hepatocyte homogenates, 10 μM dicumarol. After preincubation at 37° C. for 1 min, reactions were started with 1 mM NADPH and incubated for 5 min. After adding 0.25 ml of cold acetone and centrifugation at 3,000 rpm for 15 min, resorufin was measured as above using the spectrophotofluorimeter (Rifkind et al., 1994).

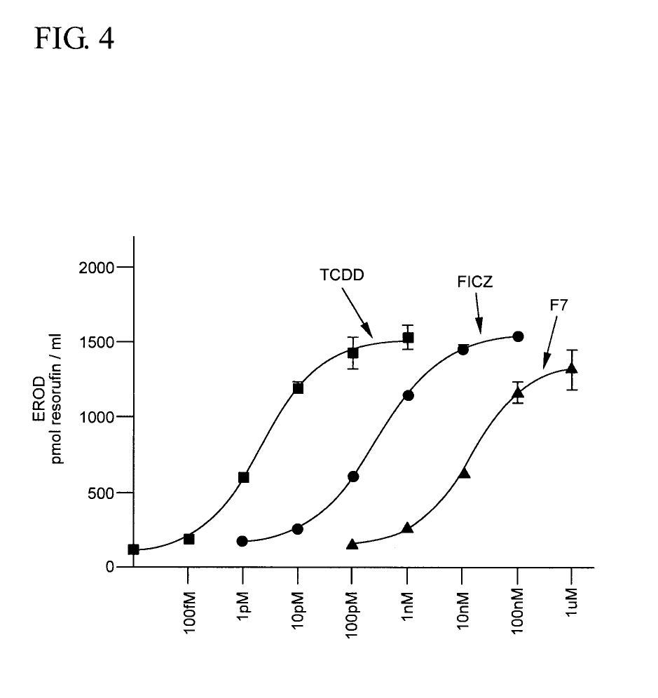

Data for Compound F7 is shown in FIG. 4. Data for Compounds F4 and F5 are not shown but had similar activity.

Glucose Production Assay.

Primary hepatocytes were cultured in six-well plates (1.4 times 106 cells per well) in DMEM with 10% FBS or, in case of hormonal treatments, in serum-free DMEM. The medium was then replaced with 1 ml of glucose production buffer consisting of glucose-free DMEM (pH 7.4), without phenol red, supplemented with 20 mM sodium lactate and 2 mM sodium pyruvate. After a 3-h incubation, 0.5 ml of medium was collected and the glucose concentration measured with a colorimetric glucose assay kit (Sigma). The readings were then normalized to the total protein content determined from the whole-cell lysates. (Yoon J C, et al. Nature 413: 131-8, 2001.)

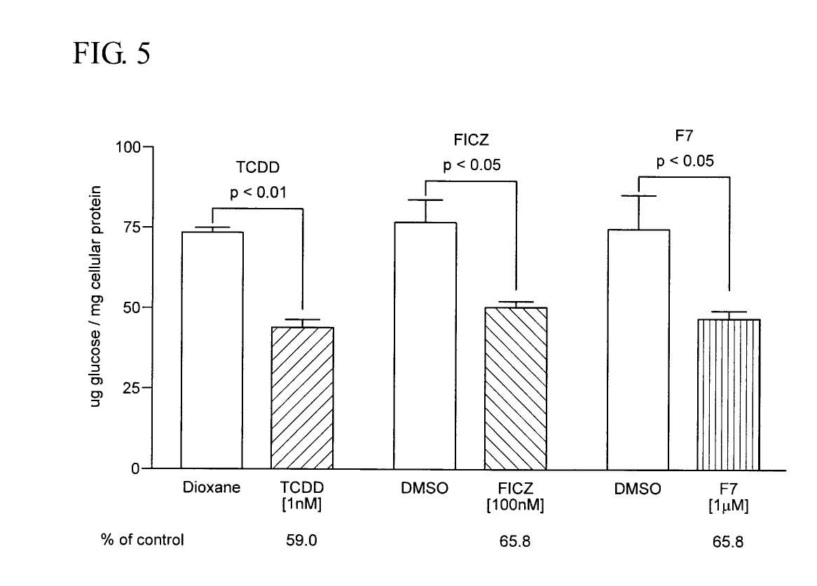

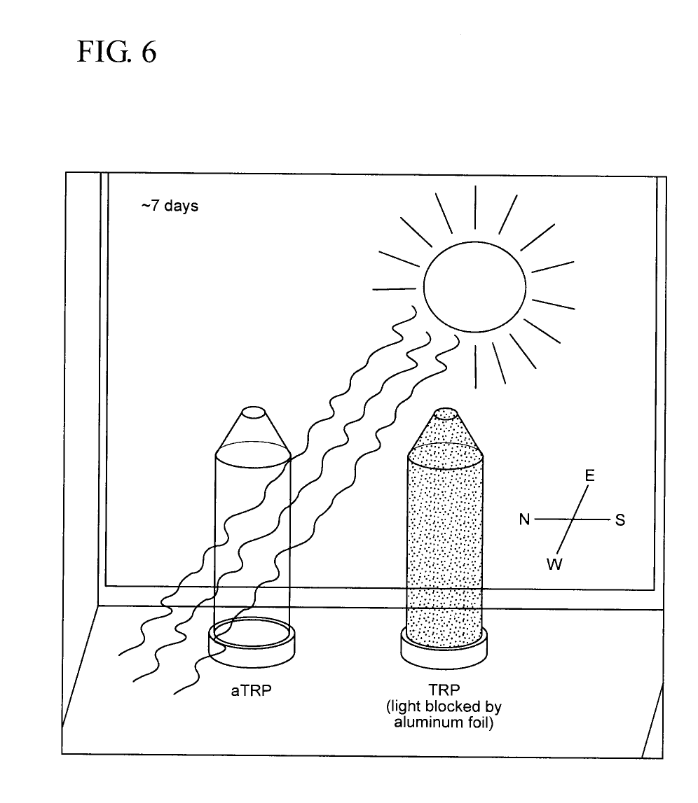

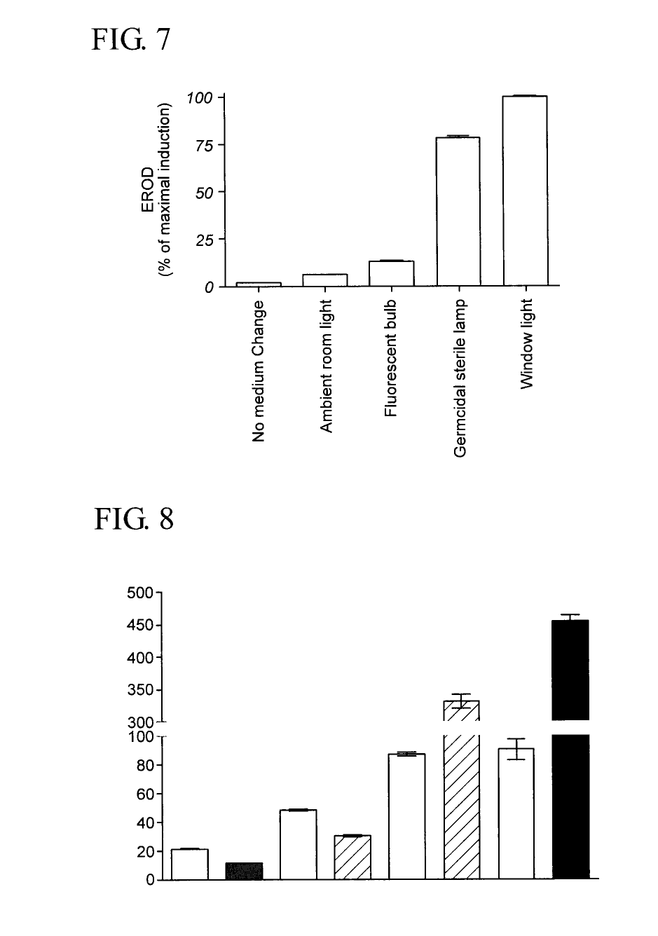

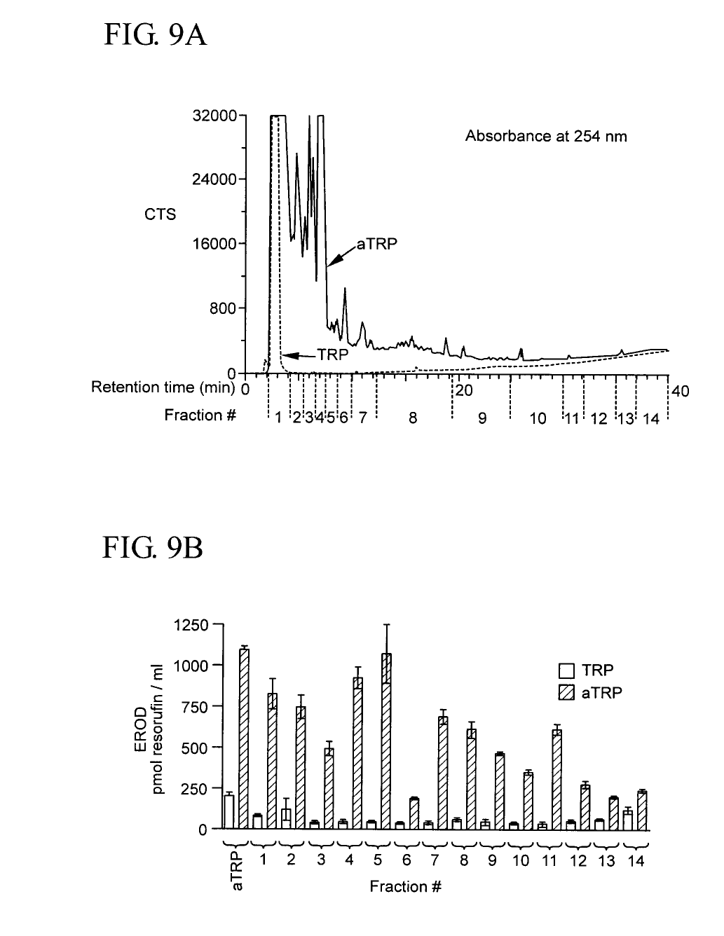

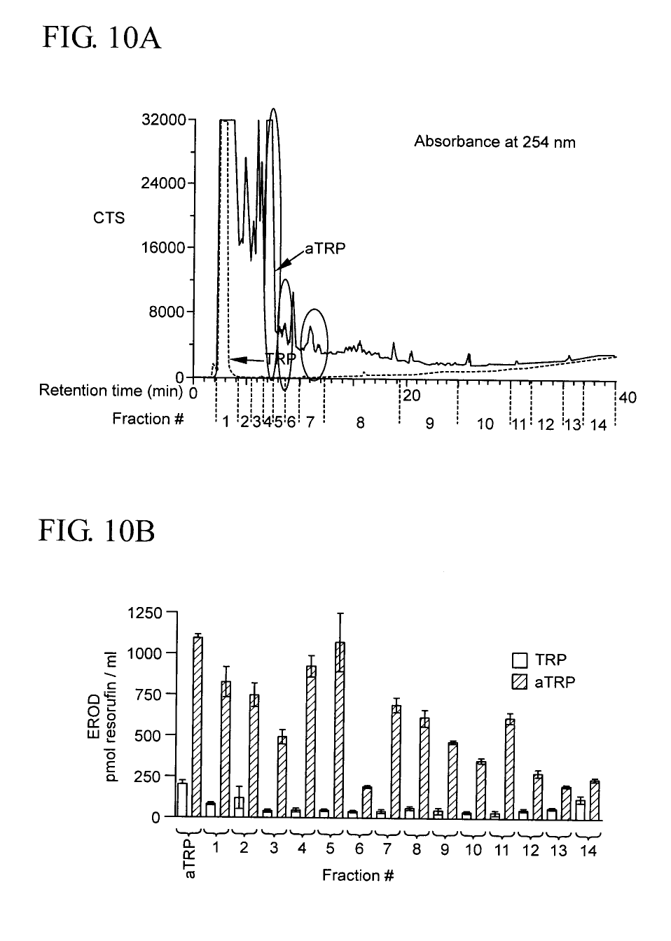

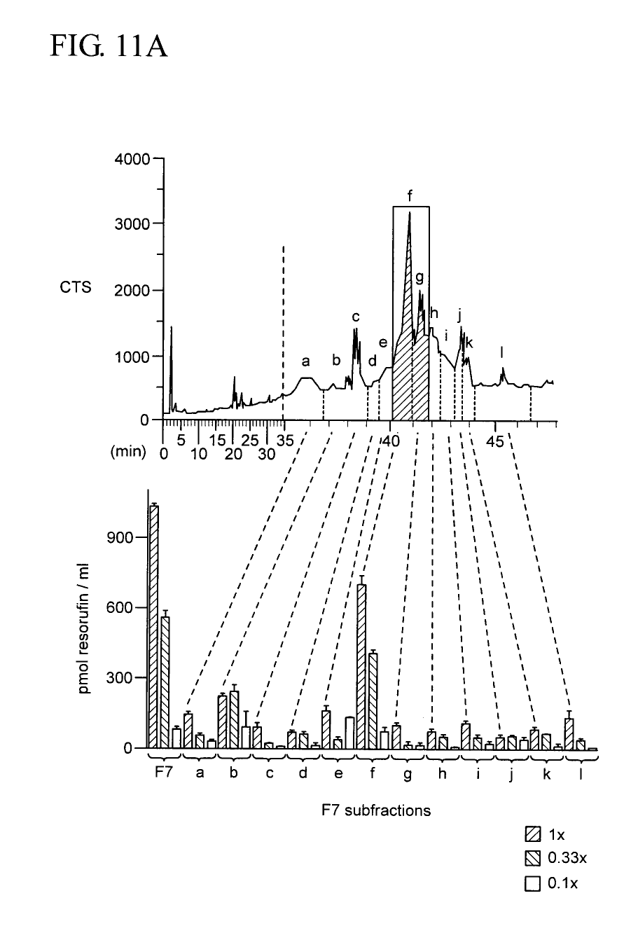

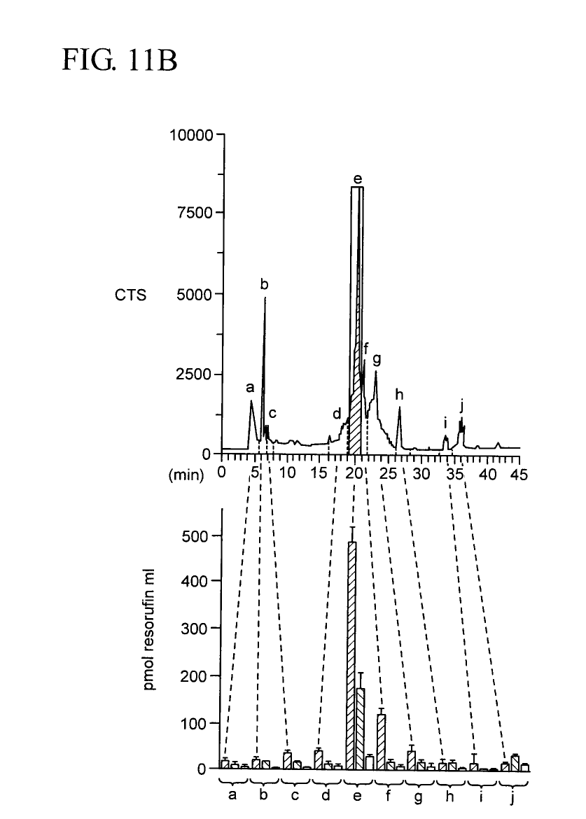

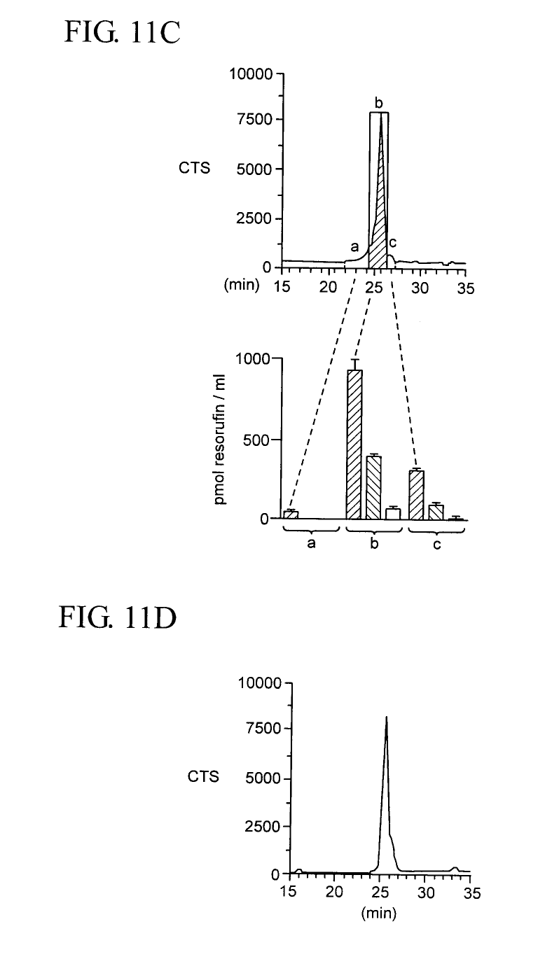

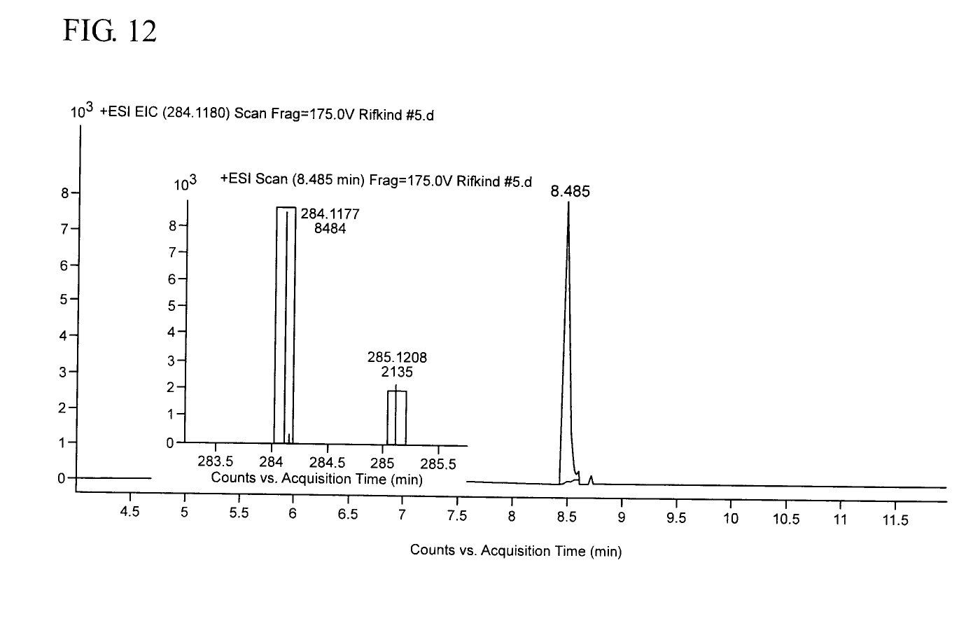

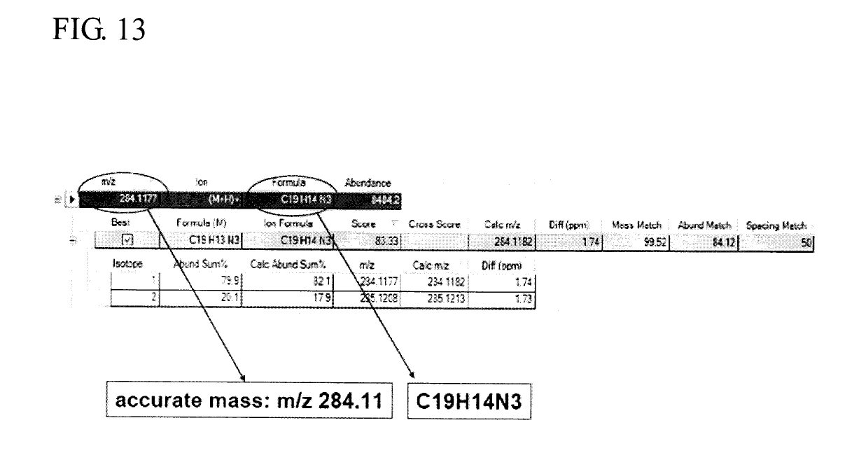

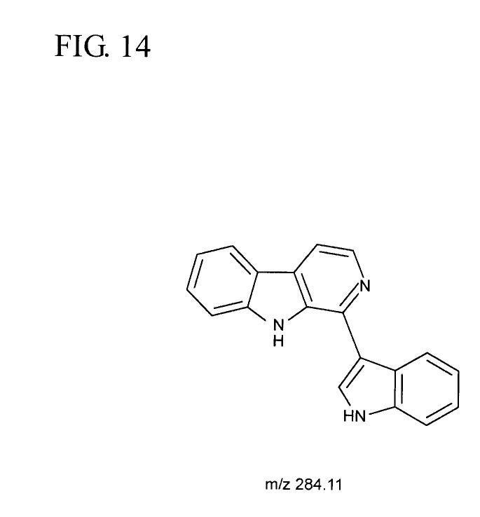

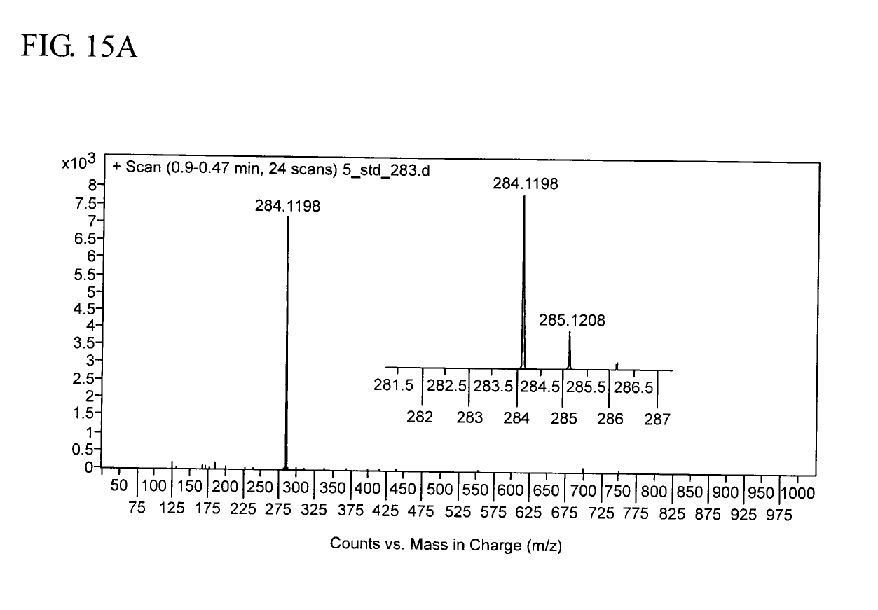

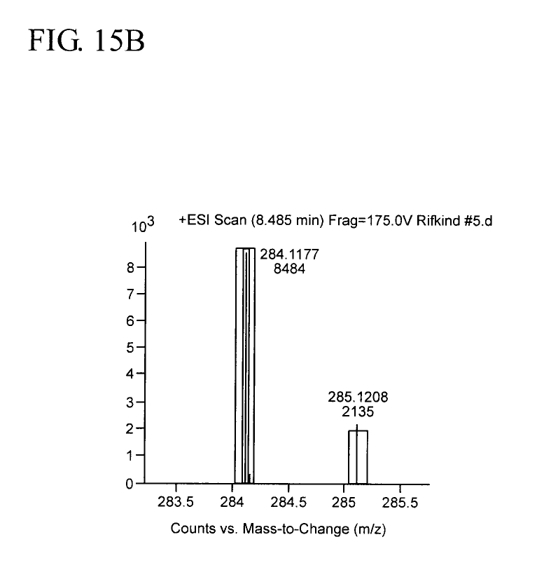

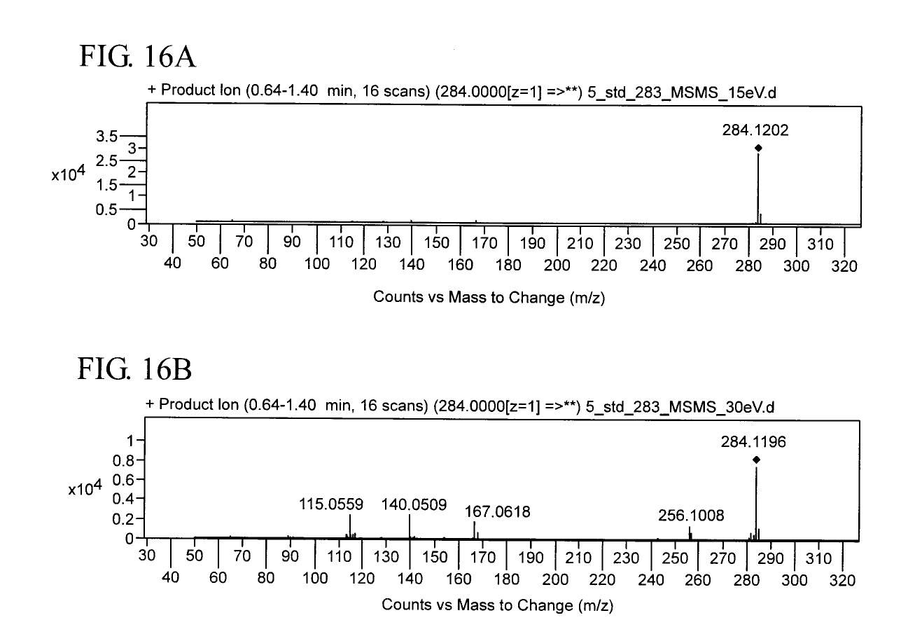

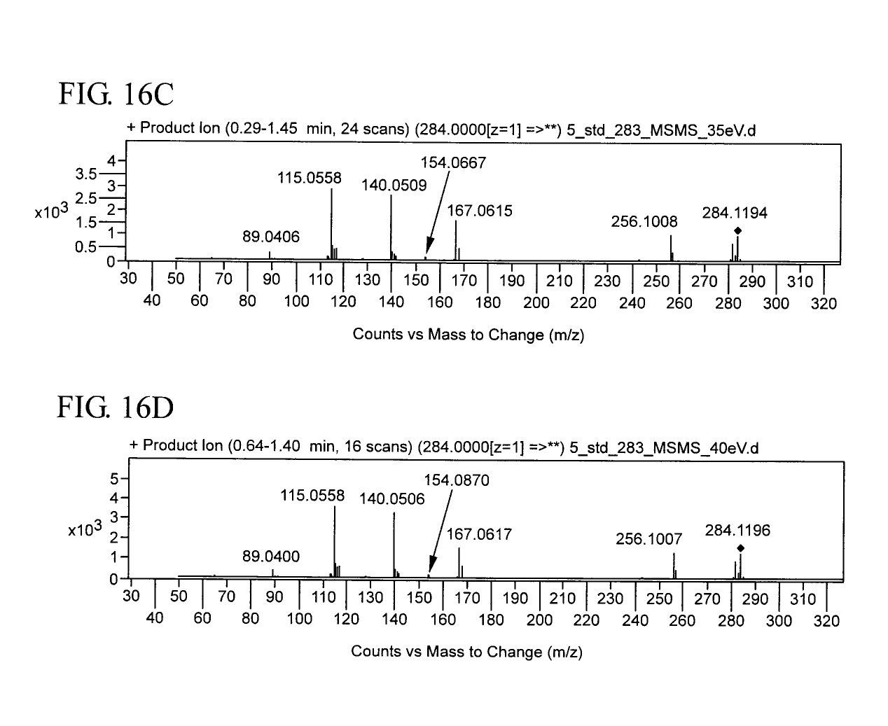

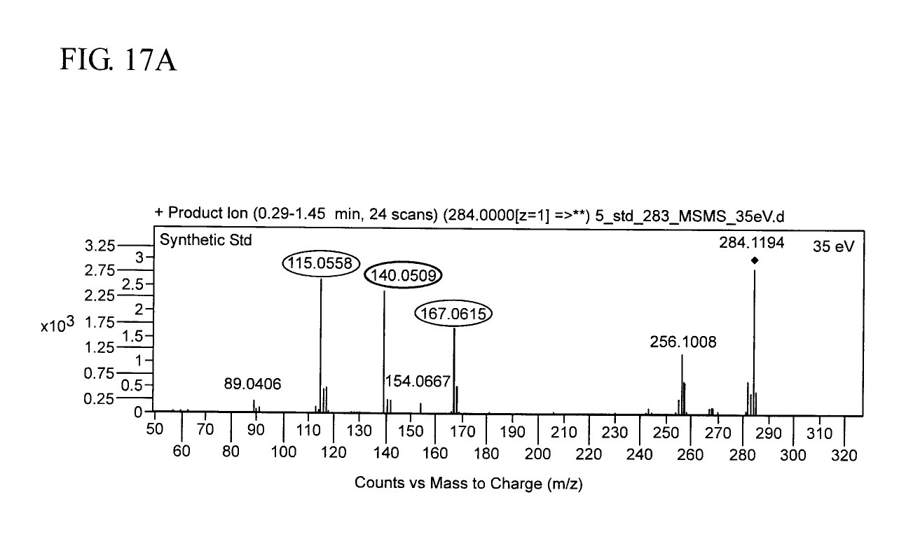

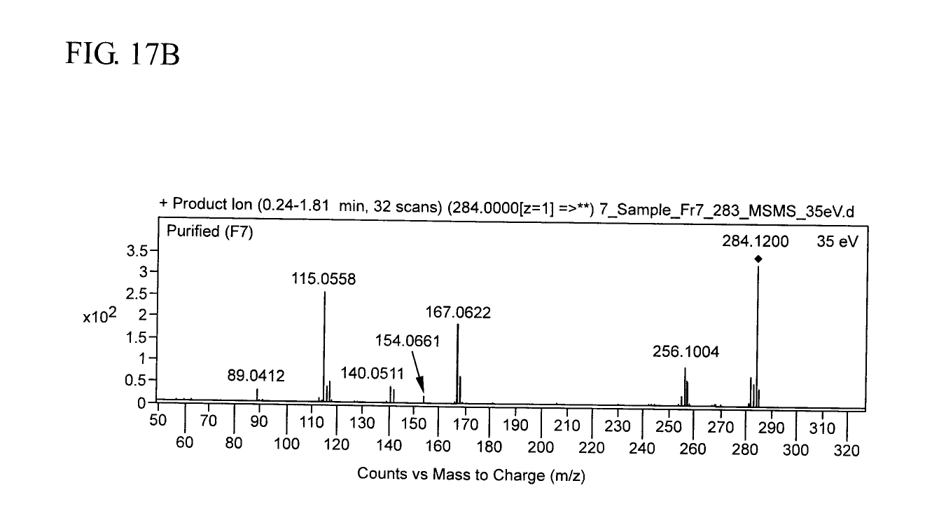

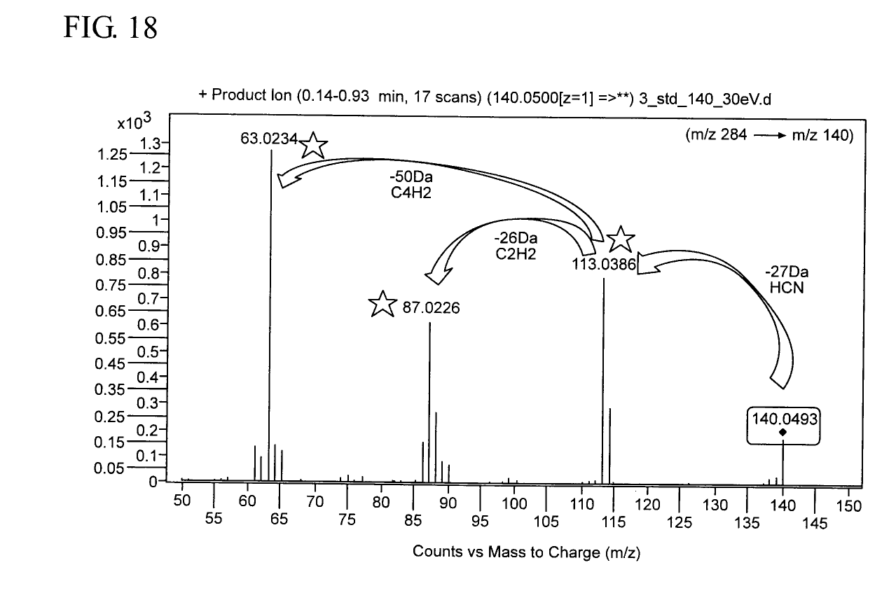

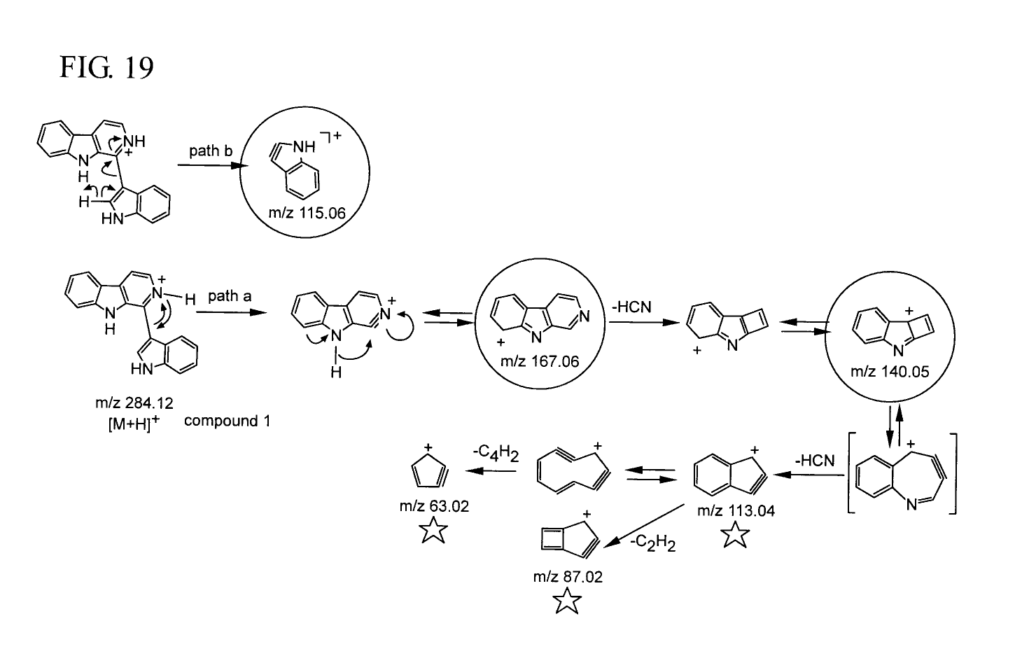

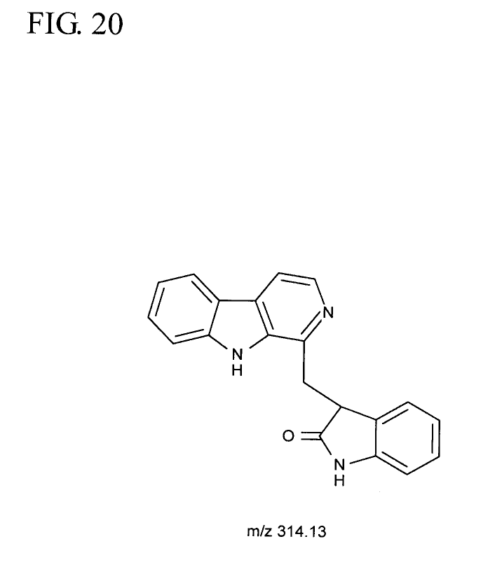

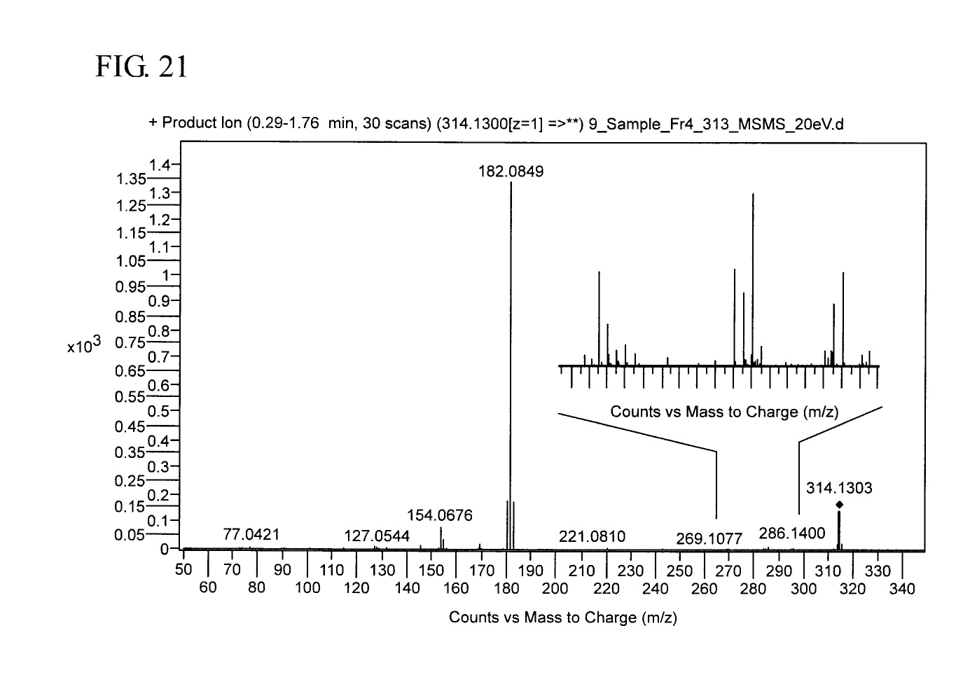

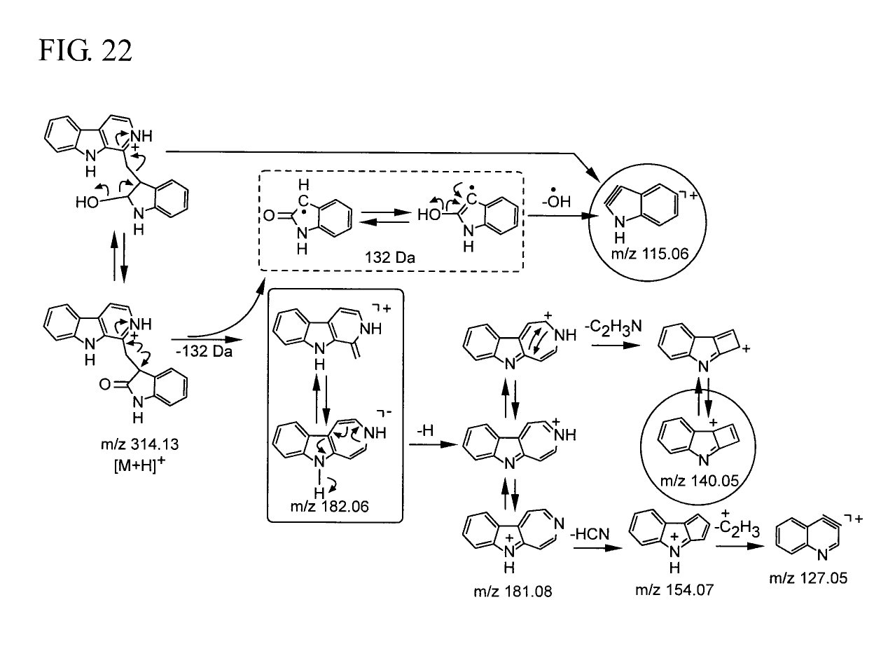

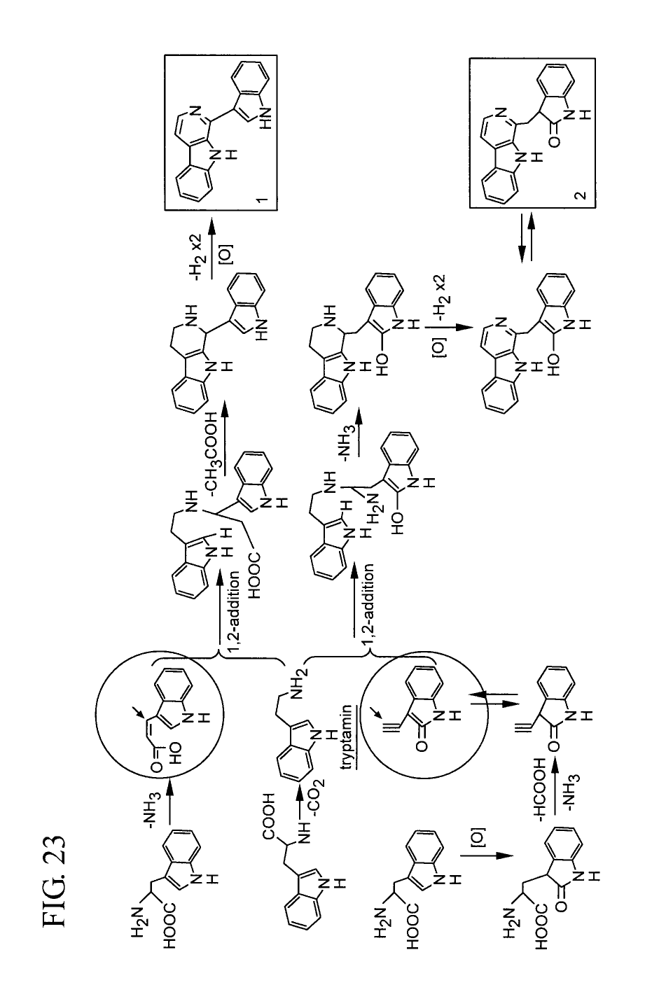

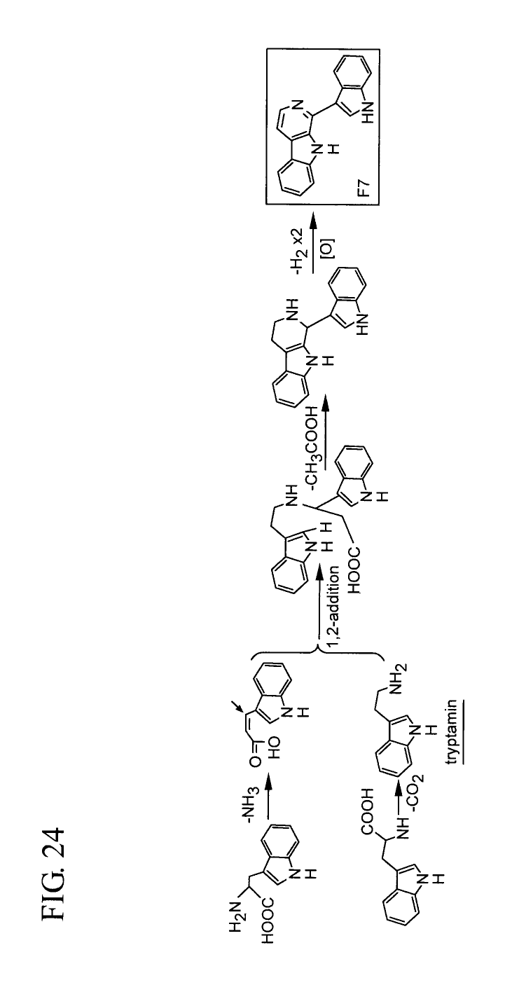

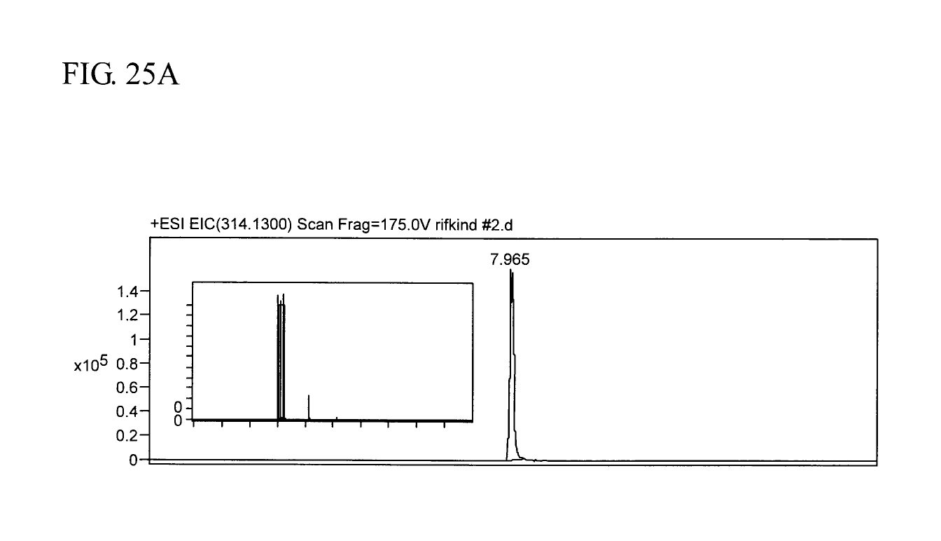

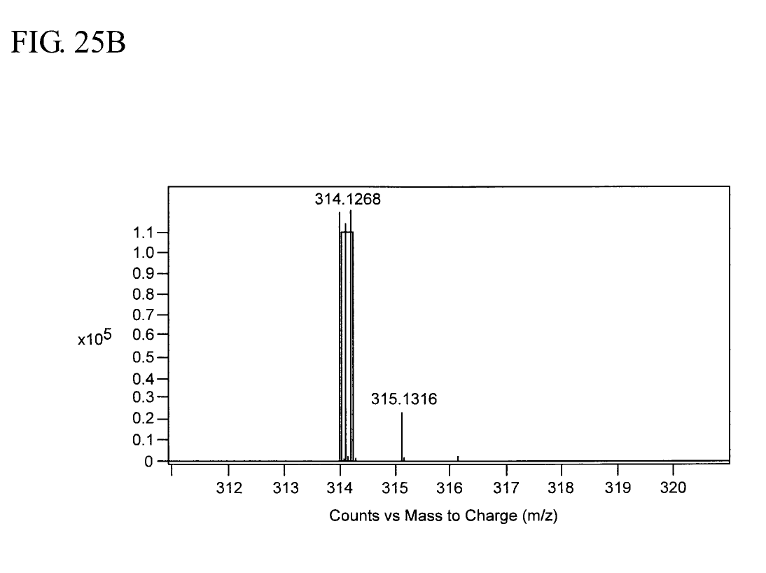

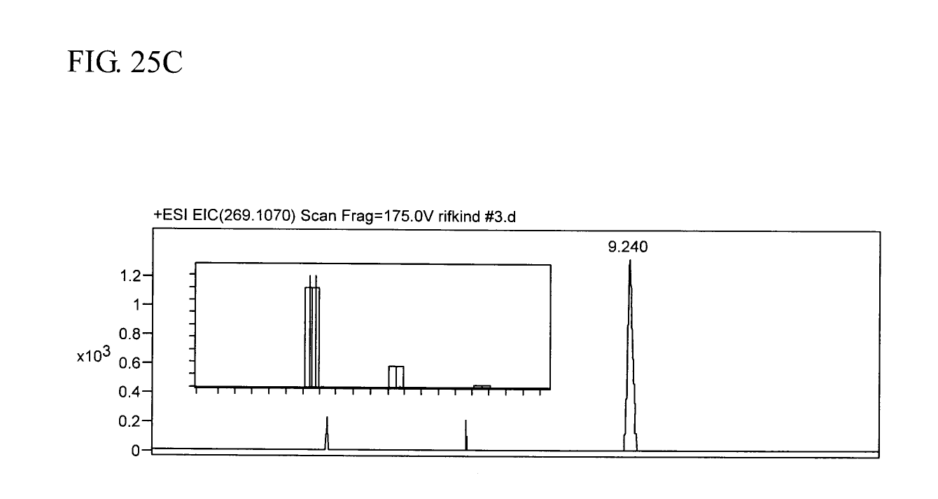

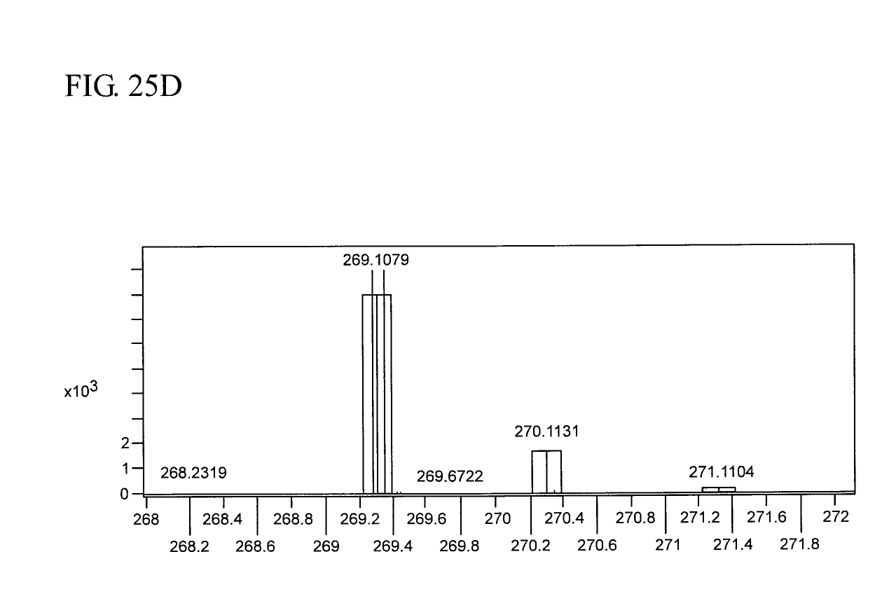

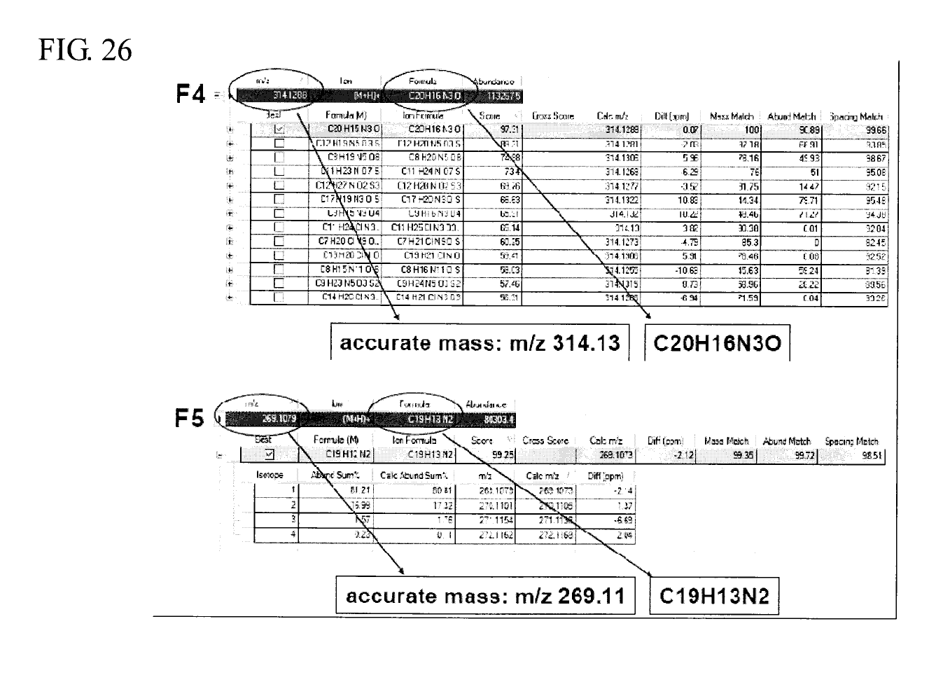

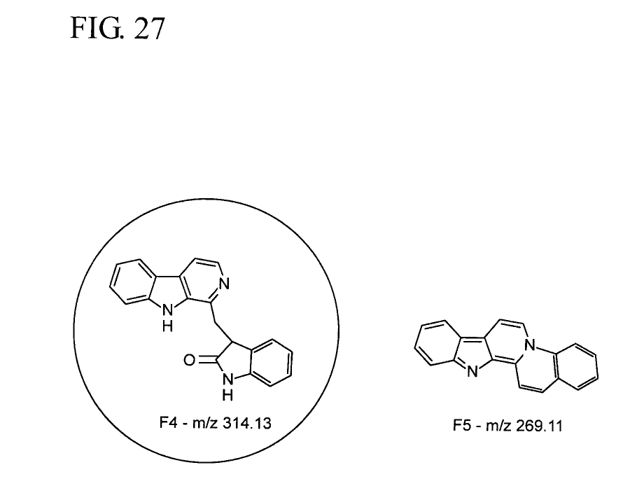

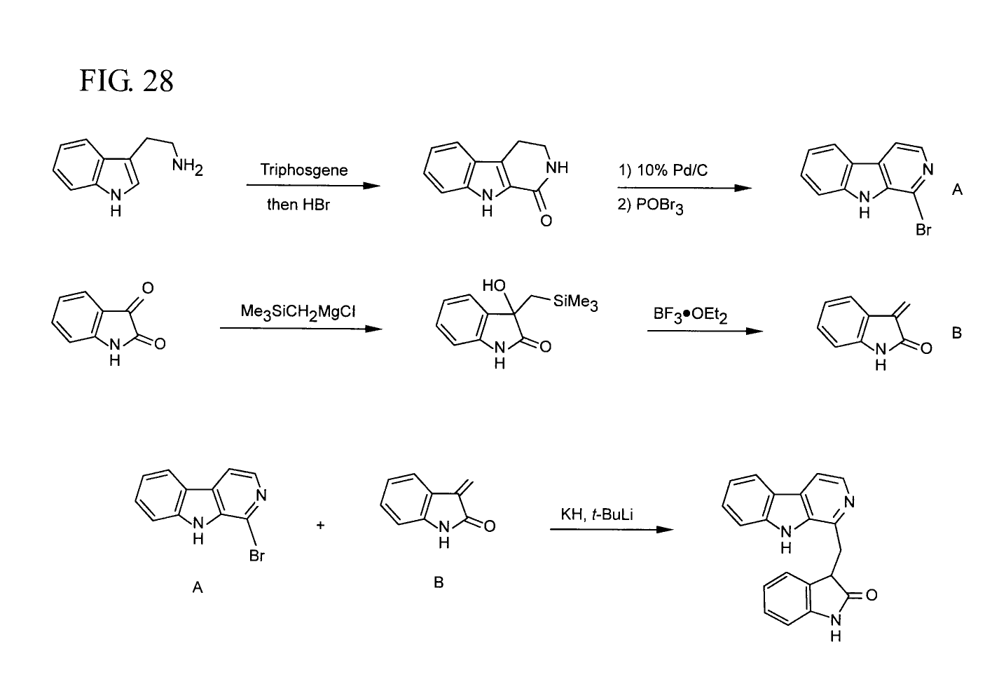

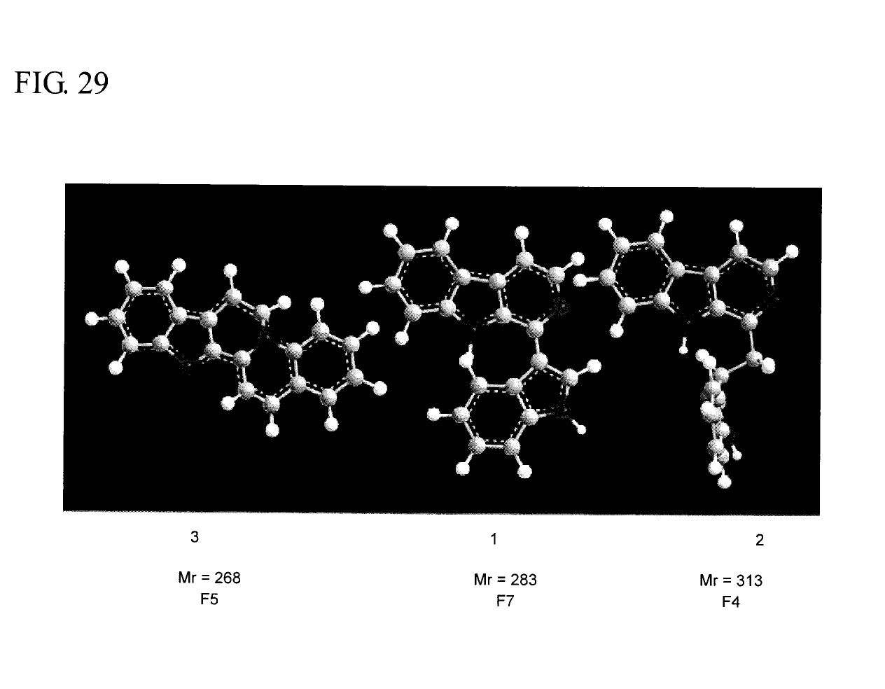

Data for Compound F7 is shown in FIG. 5. Data for Compounds F4 and F5 are not shown but had similar activity.

Isolation of Most active fractions, and most active compound within each fraction, and identification of formula and structure. See Figures

The following statements are potential claims that may be converted to claims in a future application. No modifications of the following statements should be allowed to affect the interpretation of claims which may be drafted when this provisional application is converted into a regular utility application.

### REFERENCES

Rifkind, A. B., Kanetoshi, A., Orlinick, J., Capdevila, J. H., and Lee, C. (1994). Purification and biochemical characterization of two major cytochrome P-450 isoforms induced by 2,3,7,8-tetrachlorodibenzo-p-dioxin in chick embryo liver. J. Biol. Chem. 269, 3387-3396.

Yoon J C et al. Control of hepatic gluconeogenesis through the transcriptional coactivator PGC-1. Nature 413: 131-8, 2001.

What is claimed is:

1. An isolated compound represented by the following formula: 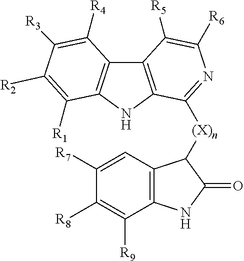
Wherein
X is alkyl or a heteroatom; ‘n’ may be an integer from 1 to 10. Xₙ is cycloalkyl, heterocycloalkyl, cycloalkenyl, heterocycloalkenyl, aryl, heteroaryl, aralkyl, or heteroaralkyl;
each R group may independently be hydrogen, a heteroatom, cycloalkyl, heterocycloalkyl, cycloalkenyl, heterocycloalkenyl, aryl, heteroaryl, aralkyl, or heteroaralkyl;
the R-groups may be linked to form cycloalkyl, heterocycloalkyl, cycloalkenyl, heterocycloalkenyl, aryl, heteroaryl, aralkyl, or heteroaralkyl rings.

2. The compound of claim 1, wherein
X is carbon, and n=1; and
each R-group is H

3. A method of modulating AhR by administering a composition comprising an isolated compound of claim 1.

4. A method of treating a proliferative disease by administering to an animal in need thereof a compound of claim 1.

5. A method of treating a metabolic disease by administering to an animal in need thereof a compound of claim 1.

6. A method of activating AhR by administering a composition comprising an isolated compound:

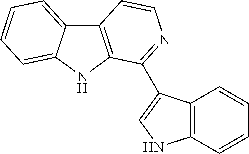

7. A method of treating a proliferative disease by administering to an animal in need thereof a composition comprising an isolated compound:

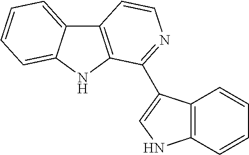

8. A method of treating a metabolic disease by administering to an animal in need thereof a composition comprising an isolated compound:

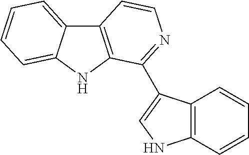

9. A method of activating AhR by administering a composition comprising an isolated compound:

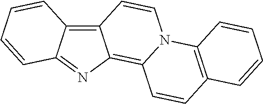

10. A method of treating a proliferative disease by administering to an animal in need thereof a composition comprising an isolated compound:

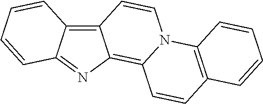

11. A method of treating a metabolic disease by administering to an animal in need thereof a composition comprising an isolated compound: [chemical structure]
